(19) United States
(12) Patent Application Publication
(10) Pub. No.: US 2018/0049684 A1
(43) Pub. Date: Feb. 22, 2018

(54) Enhanced Lancing Device with Suction

(71) Applicants: Pavol Ovcik Berresford, Centennial, CO (US); Olga Boychenyuk, Centennial, CO (US)

(72) Inventors: Pavol Ovcik Berresford, Centennial, CO (US); Olga Boychenyuk, Centennial, CO (US)

(21) Appl. No.: 15/674,482

(22) Filed: Aug. 10, 2017

Related U.S. Application Data
(60) Provisional application No. 62/377,507, filed on Aug. 19, 2016.

(51) Int. Cl. A61B 5/15; A61B 5/151; A61B 5/157

(52) U.S. Cl. CPC A61B 5/150099; A61B 5/150175; A61B 5/157; A61B 5/15111; A61B 5/150358

(57) ABSTRACT

A lancet device is formed of an outer housing that provides a source of vacuum. An inside, nested lancet device may provide lancet blade cocking, locking, and release functions. A forward shell can be pressed against the user's skin to thereby seal and establish a vacuum chamber around the lancing site. A sample collection strip resides at the lancing site to receive the produced blood sample. A sensor reads data from the sample and communicates with an internal processor to manage and communicate the data. The collection strip has multiple sectors serving as separate sampling areas that can be sequentially repositioned to an active location in line with the lancet blade. Each sampling area has a predefined hole in line with the path of the lancet blade so that the blade can cleanly traverse the sampling area.

### BACKGROUND OF THE INVENTION

### Field of the Invention

The invention generally relates to surgery and to collecting a liquid from the body. More specifically, the invention relates to improvements in housing and operating a lancet, which is a sharp edge or point for piercing the skin to draw blood. Further, the invention relates to an improved device for use in cutting, puncturing, or piercing the body or a specific portion thereof. Such piercing is by a lancet or similar instrument with a short, wide, double edged, pointed blade that cuts as the instrument is advanced longitudinally, or a blade that can repeatedly produce an incision of specific length, or an incision of specific length and of specific depth in body tissue. The invention further relates to a device employing a lancet blade that is actuated from a checked or inoperative position when released to an operative position by an energy storing resilient means. The invention is especially applicable to applying user-controlled variable suction or vacuum to skin area adjacent to and surrounding a pierced location.

### Description of Prior Art

To monitor and treat various diseases and conditions such as diabetes, taking a blood sample is required of many people, often numerous times during a single day. Instruments known as lancet devices provide a quick means for producing a sample of blood for analysis. A basic lancet device is a housing with a longitudinally slidable holder for a puncturing element at a forward or distal end thereof. Different styles of puncturing elements can be used, including needles and blades. For convenient reference, the puncturing element will be generally referred to as a blade without limiting the scope of puncturing elements included therein. The puncturing element holder or blade holder can receive and carry a disposable puncturing element or lancet blade with the blade extending forward. As an aid to initial placement of the lancet device on the user's skin, a nose or spacer element at the forward or distal end of the housing is suitably long to space the housing, lancet blade holder, and lancet blade from the user's skin when the spacer is pressed against the skin prior to triggering. The lancet device can be cocked to place the lancet device in a ready condition for the blade to be released or triggered to spring forward from the spacer and pierce the user's skin. A basic lancet device contains an actuator to drive the lancet blade forward. For example, a mechanical cocking element can compress a spring that will drive the lancet holder forward when released. The cocking element also may employ a checking element to lock the lancet holder in a checked position, awaiting release.

When released, the blade moves forward and pierces the user's skin. The tip of the blade will extend forward from the distal end of the spacer. A trigger element associated with the housing can be actuated to release the lancet holder from checked position; whereupon the spring will cause the lancet holder to shoot forward, rapidly sliding the lancet blade forward and through the open end of the spacer to pierce the user's skin where the open forward end of the spacer is pressed against the skin. The spring or other driving actuator allows the blade to pierce the user's skin and then withdraw. For example, a spring actuator may allow the blade to continue forward by inertia, beyond a position of neutral spring forces, such that after puncturing the user's skin, the blade springs back from its maximum forward position. The maximum forward extension beyond a location of neutral forces represents the depth of skin penetration. Thus, lancet devices of this type are able to pierce the user's skin so rapidly, and with controlled depth, that there is little discomfort, producing a drop of blood for sampling.

Variations in the features of lancet devices provide differences in performance. A lancet device can be constructed to apply vacuum to the piercing site by incorporating a vacuum chamber in the housing, behind the lancet holder. In such a lancet device, a spring-loaded piston can be checked in a forward position. When triggered to be released, the piston shoots backwards, thereby creating a vacuum or low pressure volume in the vacuum chamber. At the same time, the lancet blade is advanced to pierce the user's skin similarly to the previous description. However, this device differs because the vacuum is applied through the space to the pierced skin area surrounding the lancet. The vacuum is maintained due to the seal established between the forward end of the spacer and the user's skin. The trigger defines a vent hole that is covered by the user's finger during triggering operation. By moving his finger from the vent hole, the user can open the vent and dissipate the vacuum before attempting to remove the lancet device from the user's skin.

Some users have found that the fixed vacuum of such a lancet device is stronger than they wish to experience. Also, users who might like the advantages of a vacuum feature may find that the cost of the vacuum feature is too high. The basic lancet devices, without any vacuum pump, tend to be far more economical.

It would be desirable to achieve the economy of a basic lancet device while providing access to a vacuum feature, as desired. Maintaining a low cost in a medical device is widely important and desired, especially among frequent users of such a device.

It would be desirable to allow the user or patient to vary the degree of vacuum according to his own needs. In many cases, the user and the patient are the same person, who then can judge when vacuum is sufficient. In other cases, such as with a parent who assists his child, the parent is the user. The parent and child can determine between them how much vacuum is desired or needed.

To achieve the foregoing and other objects and in accordance with the purpose of the present invention, as embodied and broadly described herein, the method and apparatus of this invention may comprise the following.

### SUMMARY OF THE INVENTION

The accompanying drawings, which are incorporated in and form a part of the specification, illustrate preferred embodiments of the present invention, and together with the description, serve to explain the principles of the invention. In the drawings:

### BRIEF DESCRIPTION OF THE SEVERAL VIEWS OF THE DRAWINGS

FIG. 1 is an assembly view of a first embodiment of the lancing device with variable suction, with dashed lines suggesting an order of assembly.

FIG. 2 is a side elevational view of the lancing device with variable suction, showing the device in fully assembled configuration.

FIG. 3 (Prior Art) is a top plan view of a core lancet device.

FIG. 4 is a top plan view of a base section of an external housing, showing a vacuum pump in a cavity of thereof.

FIG. 5 is a view similar to FIG. 4, showing addition of a processor and battery to the cavity therein.

FIG. 6 is a view similar to FIG. 5, showing addition of a lancet device to the cavity therein.

FIG. 7 is a top plan view of an inverted cover section of an external housing.

FIG. 8 a top plan view of a closed external housing with a lancet device contained therein.

FIG. 9 is an isometric assembly view of an enhanced lancet device, with dashed lines suggesting an order of assembly.

FIG. 10 is an enlarged, isometric, detail view of the distal end of an enhanced lancet device, showing assembly of an external housing to a vacuum shell.

FIG. 11 is an enlarged isometric detail view of the distal end of an enhanced lancet device, showing a vacuum shell joined to an external housing of the enhanced lancet device.

FIG. 12 is an isometric view, taken from the right rear, of a vacuum shell.

### DETAILED DESCRIPTION OF THE INVENTION

A core lancet device is an elongated housing that carries at its front end a longitudinally oriented holder for a puncturing element. Typically, the puncturing element is disposable and is easily installed and removed from the holder. Various types of puncturing elements can be used with a core lancet device, including needles and blades. For convenience of reference, the puncturing element sometimes will be referred to as a lancet blade, without limiting the scope of puncturing elements included therein. A driving actuator, which will be sometimes referred to as a spring, is associated with the holder to interact as a driving member after being appropriately cocked and then released. A cocking element of the core lancet device can cock the holder or driving actuator to a checked position. A triggering element can release the holder or driving actuator from checked position. The driving actuator operates such that when the holder is released from checked position, the actuator provides a driving force that rapidly moves the holder forward. When a lancet blade is mounted in the holder, the blade springs forward with the holder. A depth control mechanism is associated with the holder to allow the blade to penetrate a user's skin to a certain depth and then to withdraw. As an example of a depth control mechanism where a blade is driven forward by a spring, the blade might continue forward by inertia, going beyond a neutral position of the spring, and then spring back to a neutral position. The excess advancement due to inertia can be how the blade pierces the user's skin. The springing back to neutral position is how the blade can withdraw from the user's skin. Thus, the piercing is rapid and depth is limited, with the entire forward piercing and rearward withdrawal taking place so quickly that the eye may have trouble seeing it.

Typically a removable, hollow tubular spacer is attached as a front end of the core lancet housing, with the path of the blade aligned to pass through the centerline of the tubular shape. The length of the spacer is such that the blade does not extend out of the open forward end of the spacer, both when the holder is in neutral position and when the holder is checked position. However, the spacer is short enough that the blade will extend out of the open forward end of the spacer when the blade is released from cocked condition and springs forward beyond neutral location. The tubular spacer enables the user to press the core lancet device against his skin to pre-position the lancet device without contacting the blade. Some lancet devices include a depth control that alters the maximum extension of the blade beyond the spacer. The length of the spacer can accommodate adjustment of this type while protecting the user from premature contact with the blade.

Even after the user's skin has been pierced, it can be difficult to bring the drop of blood to the skin surface. Sometimes the user has to squeeze and rub the pierced skin area to bring out the blood sample. The present invention provides an enhanced lancet device that improves this situation by adding suction to the function of a core lancet device that was constructed without built-in provision for suction. An aspect of the invention is the discovery that a vacuum function can be added to a core lancet device to produce an enhanced lancet device, where the core lancet device was not equipped with its own vacuum function independent of an external housing. The invention adds a coordinated source of vacuum, suction, or low pressure air that is applied to a core lancet device over a selected span of time. Suction might be applied immediately prior to the skin piercing event in order to build up suction strength. Suction might be applied during the piercing event, itself, to preserve previously built up suction and to extract blood. Suction might be applied immediately after the piercing event to extract the sample of blood.

This invention is primarily directed to a core lancet device that is nested in an external vacuum housing. The term, “core,” refers to a lancet device or housing that is not especially configured to generate vacuum. Such a core lancet device is characterized by an absence of an internal vacuum chamber. A functional fit between parts can allow an external chamber to surround the core lancet with vacuum, other than at a forward nose near the holder for the lancet blade. The external vacuum establishes air seepage through the core lancet device from the forward nose, creating suction in proximity to a forward puncturing device or blade. The external vacuum chamber houses the core lancet except at the forward nose, which serves as a suction inlet point. Thus, in an enhanced lancet device, an external vacuum housing operates through a core lancet housing to draw suction in proximity of the lancet blade. When a spacer is attached to the forward nose, the suction inlet point is advanced to the front end of the spacer, which applies the suction to the user's skin where the lancet blade will pierce the skin. Thus, the open front of the spacer is an extension of the forward suction point. The internal low pressure air from the external housing and internal lancet housing draws in the higher pressure atmospheric air at the front nose of the lancet housing and around the lancet blade holder, creating external suction that is applied to the user's skin through the spacer.

In greater detail, the invention is an outer vacuum housing that contains a core lancet device or core lancet housing. The outer vacuum housing is substantially pressure sealed, other than through the core lancet housing. The core lancet housing serves as an inner housing contained within the outer vacuum housing so that the inner housing is substantially surrounded by low pressure or vacuum, other than at the front where the blade holder and lancet blade are located. The combined inner and outer housings provide variable vacuum or suction and, in use, apply it through a tubular spacer to a user's skin in the area that the lancet blade will pierce.

The front of the inner core lancet housing and the front of the external vacuum housing meet at a forward junction. The inner and outer housings can be fastened together at the junction. A suitable junction area of the core lancet housing is an outward facing cylindrical forward end that encircles the lancet holder. A suitable junction area of the outer housing is an inward facing cylindrical forward end that closely fits around the junction area of the core lancet device. The two junction areas are placed at the same end of the combined inner and outer housings and are aligned with the pathway for the lancet blade.

The two junction areas have complementary shapes to establish the junction at a perimeter around the position of the lancet blade holder so that the lancet blade can move forward to puncturing position without interference from the junction. The core lancet provides the inner side of the junction, while the external vacuum housing provides the outer side of the junction. The junction minimizes or eliminates vacuum leakage between the two mating structures.

A tubular spacer or shell is formed or attached to the front end of the enhanced lancet device, at the junction between the inner core lancet housing and the external vacuum housing. This junction encircles a suction point. The spacer or shell provides a lateral wall around the path of the lancet blade to bring suction forward, as needed, to apply suction to the user's skin in front of the cocked lancet blade. The spacer or shell is pressed against the user's skin to establish a known spacing to the cocked lancet blade. Then, when the trigger element releases the holder and blade, the blade advances on its path by a known distance that is sufficient for the blade to penetrate the user's skin. However, the known distance of blade travel is limited, so that penetration is sufficient for blood sampling but not needlessly deep into the user's skin. The spacer or shell provides both an application of suction to the penetration site and a limitation of how deep the blade can penetrate beyond the forward end of the spacer or shell.

According to the invention, a core lancet housing can serve as an inner housing that is nested within the outer or primary housing, with the exception that the blade holder and lancet blade are aligned with a passage to the outside. The outer housing serves as a vacuum chamber and cooperates with the inner core lancet housing to supply suction into the outside passage. The suction or vacuum is transmitted through the outside passage, which may laterally confine the suction through a tube or shell to reach a forward position at the user's skin. The tube or shell become sealed to apply suction against the user's skin when pressed against the user's skin, and as a result, the suction or vacuum then is applied to the user's skin at the location where the lancet blade will pierce it. The applied suction enhances extraction of a drop of blood for sampling. After use, typically the tubular spacer or shell is removed from the enhanced lancet device to gain access to the blade holder and the lancet blade is replaced. The spacer is reattached to prepare the enhanced lancet device for a next use. Several embodiments demonstrate the scope of this invention.

According to a first embodiment, the invention can be adapted for use with a core lancet housing of known configuration but lacking a vacuum feature. In such a version, an otherwise complete core lancet housing is nested inside a cavity within an external housing, which then is sealed against loss of vacuum other than through a portion of the lancet housing with outside access, such as within the junction. The sealed cavity provides a basis for applying suction or vacuum around substantially the entire lancet device, other than the junction. Reference to a sealed external housing or sealed cavity also allows for air access to the open tip of the spacer. Otherwise, the enhanced lancet device is sealed when pressed against the user's skin. The reference to a sealed external housing or sealed cavity also allows for discharge of air through an exit port from a source of suction to establish a zone of lower pressure in the cavity.

In order to provide vacuum to an internal nested lancet housing, the external housing is configured with a combination of vacuum resistant walls and access areas. The pre-existing controls of the core lancet housing are operated by limited deformation of the access areas. Access area covers and shrouds provide such desired deformation to allow operation of controls on the lancet housing. The access area covers sufficiently resist deformation under vacuum that a vacuum can be applied to a lancing location on the user's skin.

According to an alternate embodiment, the invention is adapted to a sealed primary housing that provides the functions and equipment of a lancet device. These are a sliding lancet carrier or holder, a resilient member such as a spring loaded against the sliding lancet holder, a cocking element for withdrawing the sliding lancet holder to withdrawn position, a check element for locking the sliding holder in withdrawn position, and a trigger element for releasing the lancet holder from withdrawn position to slide forward through a front opening, lancing the user's skin at the front opening. The invention is adapted to the primary housing by providing an access port and an external suction tube mounted to the port, enabling the user to draw a variable degree of vacuum within the sealed primary housing.

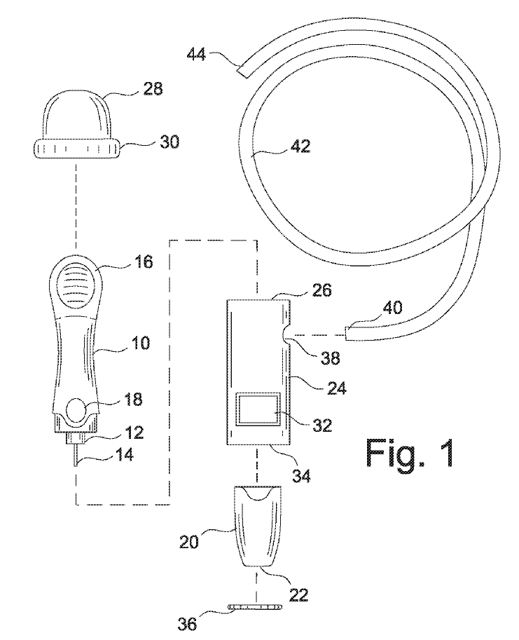

With reference to FIG. 1, an internal core lancet device is combined with an external housing. The internal core lancet device is formed of an elongated internal housing 10 that provides a longitudinally slidable lancet holder 12 that carries a disposable lancet blade 14. A cocking element 16 places the lancet holder in ready position to spring forward within housing 10 to bring the lancet blade forward. A check element inside housing 10 engages to hold the cocked lancet holder in ready position. A trigger button 18 is pressed to release the lancet holder from the check element, resulting in the lancet holder's shooting forward with lancet blade 14 to pierce the user's skin. A tubular spacer 20 fits over the forward end of housing 10. The forward end 22 of spacer 20 is open to allow the lancet blade 14 to extend out of the spacer to pierce the user's skin.

An external housing 24 is sized to receive internal housing 10. According to this specific embodiment, the cocking element 16 protrudes above a proximal or rearward open end 26 of housing 24. A flexible cap 28 fits onto rearward end 26 of external housing 24 and over cocking element 16. A retention band 30 secures the cap 28 around housing end 26 and secures a seal between the housing 24 and cap 28. The cap can be attached to housing 24 by any of various means, including pressure, adhesive, threads, or twist-lock (bayonet), to name several examples. Flexibility of the cap allows the user to pull cocking element 16 rearward to cock lancet holder 12 to checked position. If necessary, cap 28 can be formed of stretchable material to accommodate sufficient rearward movement of cocking element 16.

Housing 24 receives trigger button 18 behind window 32. The window is formed or covered by a panel that is flexible enough to allow selective operation of the button. The material of window 32 may be a soft plastic that is applied to the housing 24 to seal the window. As examples, the window panel can be set into the window opening, bonded around the window opening, or overlaid on the window opening, which can include the application of a ring of plastic that fits snugly around the housing 24. Thus, the controls on the internal housing 10 are operable behind elastic, flexible, or otherwise accessible covers while the internal housing 10 is installed within the external housing 24.

At the forward, distal end 34 of housing 24, the spacer 20 is inserted into housing end 34. The spacer typically is a component of the internal core lancing device and fits snugly onto the distal end of internal housing 10. A seal 36 is applied to spacer 20 in a position to also seal with housing 24 at lower end 34.

A port 38 is in communication with the inside volume of housing 24. The port is sized to engage with an end 40 of a suction tube 42. The opposite end 44 of the suction tube is suitable for engagement with a source of suction. The invention contemplates that the user will apply suction by mouth, so that the degree of suction is instantaneously variable at the user's discretion.

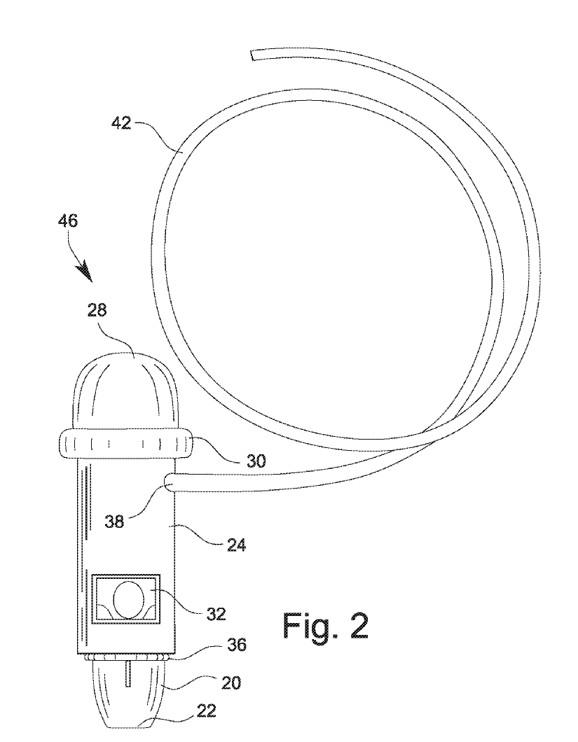

According to FIG. 2, the internal core lancet housing 10 nests within the assembled enhanced lancet housing 46 as a part thereof. In use, suction is applied through tube 42 to create a vacuum within housing 24. In use, the open end 22 of tubular spacer 20 is sealed against the user's skin. Thus, in use, the enhanced lancet device 46 maintains vacuum according to the user's control through tube 42. When the user wishes to release vacuum, he releases his suction on tube 42 and the enhanced lancet device 46 returns to ambient internal air pressure.

FIGS. 1 and 2 also suggest a configuration for a vacuum lancet device employing a suction hose 42, without the need for an internal, nested lancet device. Sliding lancet holder, cocking device, and trigger device can be built into housing 24, which is served by variable vacuum through tube 42 and port 38.

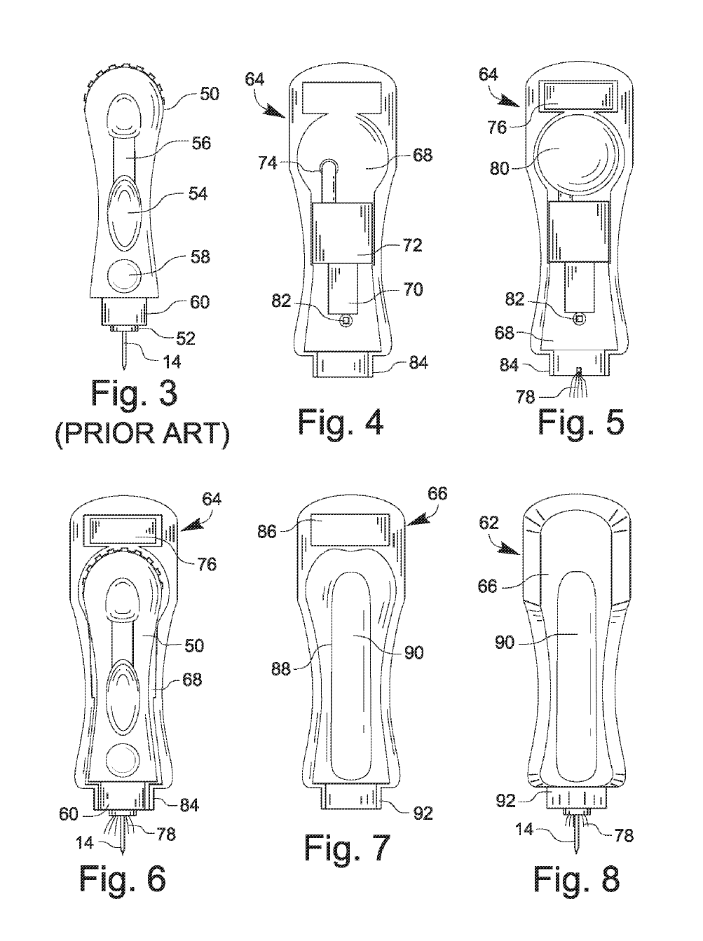

Several different core lancet housings are commercially available. FIGS. 3-8 show the concept of an external vacuum housing that can be applied to different commercially available core lancet housings 50. Reference to a core lancet housing 50 refers to a core lancet device without necessarily including a front spacer 20 or the like that was a component of the core lancet device. A spacer or vacuum shell is employed in embodiments including the external vacuum housing, but this spacer may differ from the spacer that is part of the core lancet device. In particular, the original spacer 20 may have attached directly to the nose of a core lancer device, while a spacer used with an enhanced lancet device may differ by attaching directly to a nose of the external vacuum housing.

The internal housing 50 of FIG. 3 provides a longitudinally slidable lancet holder 52 at the front end thereof for receiving and carrying a disposable lancet blade 14. Conventionally, a biasing element 54 cocks or otherwise readies the lancet holder 52 for forward movement to a puncturing position. The biasing element 54 places the lancet holder in pre-actuated condition. The biasing element might include a spring or system of springs to spring-load the lancet holder to slide forward and puncture the user's skin with the tip of lancet blade 14. While springs are a suitable driving force for a biasing element, other biasing means might be used. For example, magnets and resilient bands are other suitable choices. In one embodiment, a cocking slider 54 operates in a lateral slider slot 56 to slide rearward to a checked position, placing the lancet holder 52 in pre-actuated condition to spring forward when released. Typically a check element inside the housing 50 engages to hold the lancet holder 52 in cocked, pre-actuated position. A releasing means such as trigger button 58 is pressed to release the check element, placing the lancet holder in actuated, puncturing condition where the lancet holder 52 springs forward with the lancet blade 14 to puncture the user's skin. A cylindrical nose 60 is located at the forward end of the lancet housing 50 and laterally encircles the lancet holder 52 when in pre-actuated position. The cylindrical nose 60 is a suitable structure for attaching an external housing 62 to the front of the lancet housing 50.

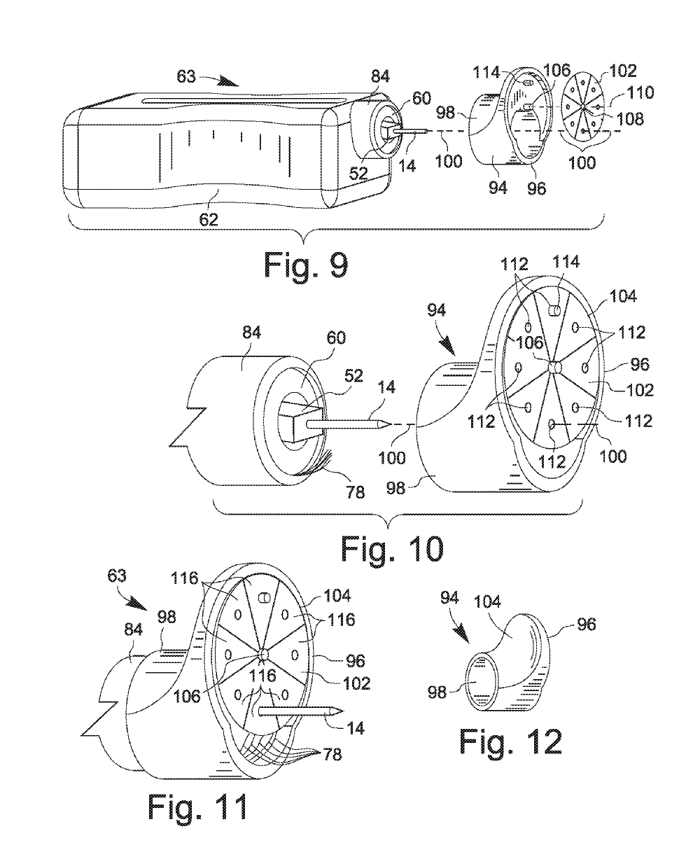

An external housing 62, FIG. 9, has an elongated, generally longitudinal shape and is sized to receive a core lancet housing 50, thus forming an enhanced lancet housing 63. The external housing opens or separates in a suitable way to receive the housing 50 and to be reassembled with the housing 50 nested within it, with the front of nose 60 open to external ambient air pressure. The housing 62 can be structured as at least two mating parts, such as a base and a cover. For example, housing 62 is composed of a base 64, best shown in FIGS. 4, 5, and 6, and a cover 66, best shown in FIG. 7. The base portion 64 of the external lancet housing 62 is formed with an internal cavity 68 that receives core lancet housing 50. The cavity 68 is illustrated as being located chiefly in the base portion 64 of housing 62, although other locations or proportions can be equally acceptable. The cavity 68 also is configured to receive supporting devices. The specific locations of the supporting devices as shown in the drawings are merely suggestive and can be altered as required or preferred in a specific configuration.

As shown in FIG. 4, one such supporting device is a means for drawing vacuum within external housing 62. The suction tube 42 allows the user to draw vacuum by suction applied to the outside end 44 of the tube. An automatic means for drawing vacuum is a micro vacuum pump formed of an electric motor 70 operating a connected pump 72. The vacuum pump draws air from within the cavity 68 and discharges the air through an exit port 74 formed in a wall of the external housing 62. When the cavity 68 is sealed by application of a cover and a junction with the core lancet device 50, the operation of the vacuum pump produces low air pressure within the cavity 68.

In addition to the vacuum pump, the housing base 64 carries a processor chip 76 that enables blood sample data to be analyzed. The chip 76 is connected to reading wires 78 that sense the content of a blood sample produced by the lancet blade 14. The chip has communication ability to manage the blood data, to convey the reading via wireless means such as cellular, wi-fi, or Internet communication to a doctor, laboratory, recording device, or other chosen recipient. A battery 80 provides power to operate the chip 76 and vacuum pump 70, 72. A manual on/off switch 82 is exposed in an external wall of the housing base 64 to enable the user to selectively operate the supporting devices in the housing 62.

The cavity 68 also is configured to receive the core internal lancet housing 50. The internal lancet housing 50 fits snugly within the cavity 68 with cylindrical nose 60 supported within a close fitting outer cylindrical ring 84 at the forward end of the housing 62. Outer ring 84 provides a sufficient seal with nose 60 so that lower pressure or vacuum can be formed inside housing 62. The ring 84 can be a part of housing 62, or it can be a separate ring that is attached to housing 62, such as by threads. Ring 84 also might be only a partial ring that is closed by a supplemental segment of the cover 66, as described below.

As best shown in FIG. 7, cover 66 fits over and closes the cavity 68 and is contoured as necessary to accommodate the intended content of the cavity 68. For example, the cover 66 might accommodate the processor chip 76 by providing a cavity portion 86 positioned over the intended location of the chip. Cover 66 might accommodate the core lancet housing 50, battery 80 and vacuum pump 70, 72 by providing a suitably contoured cavity portion 88 positioned over the intended locations of the housing 50, battery 80 and the vacuum pump 70, 72. The cover 66 also provides a slot 88 that overlies slot 56 in the lancet housing 50 and enables the controls on the lancet housing to be operated while the housing 62 remains sealed. Slot 88 may be sealed by a flexible sheet of rubber, plastic, or other suitable material 90 that enables the controls on core lancet housing 50 to be manipulated while the cover 66 remains on the base 64. Further, the cover 66 may include a cylinder segment 92, if required, to complete the outer cylindrical ring 84.

When cover 66 is applied to base 64, the housing 62 is sealed against passage of air other than within the outer cylindrical ring 84 and preferably at cylindrical nose 60. When cylindrical nose 60 is contained and sealed in the outer cylindrical ring 84, suction can be drawn through nose 60 by seepage, as present. In this way, the ring 84 serves as a perimeter of a suction port. For example, cavity 68 will be at reduced air pressure due to the operation of pump 70, 72. One possible point of entry for air is at nose 60, around the edges of the lancet blade holder 52, which is within cylindrical ring 84. Any other likely point of entry for air, such as at the junction of nose 60 and ring 84, is likewise within the perimeter established by ring 84. These possible points of seepage are desirably located within suction port 84 to supply suction in proximity to the lancet blade 14.

The use of a core lancet housing 50 contributes to the successful availability of vacuum at the lancet blade 14. A core lancet housing is not especially sealed to contain its own vacuum. Instead, the core lancet housing becomes a transmitter of suction from the external housing 62 to the forward end of the internal lancet housing 50, such as to nose 60 on the lancet housing 50. Suction at nose 60 is in surrounding proximity to the lancet blade and can be further confined and directed by a spacer or shell 94 attached to ring 84.

The external housing 62 serves as the primary vacuum chamber and relies on limited transmission of higher pressure air only within the suction port surrounded by ring 84. This suction port is partially filled by the nose 60 of core lancet housing 50, leaving seepage through the core lancet housing 50 into the external housing 62 as the transmittal path for suction through the suction port within ring 84.

It would be desirable to further confine, contain, and guide this suction to apply it to a user's skin when the lancet blade has punctured the user's skin for the purpose of extracting a sample of blood. Suitably applied suction can aid in producing the sample of blood from the puncture. In order to apply suction to a user's skin, a vacuum shell 94 is attached around and in front of the outer cylindrical ring 84 of the external housing 62. As shown in FIGS. 9-12, the vacuum shell 94 provides a smooth front contact edge 96 that can be pressed against the user's skin with sealing pressure, allowing vacuum to be applied within the perimeter established by edge 96. The back end of the vacuum shell 94 is a cylindrical ring 98 that fits over cylindrical ring 84 at the front of the external housing 62. Thus, whatever suction is available by seepage through cylindrical ring 84 is applied to the user's skin at the approximate area where the lancet blade 14 will be inserted.

In the assembled enhanced lancet device 63 of FIG. 9, the lancet blade 14 is located approximately on a common centerline of the three cylindrical rings consisting of the lancet housing nose 60, the outer cylindrical ring 84, and the shell cylinder 98. This common centerline of the three cylindrical rings is indicated by the dashed line 100 in FIGS. 9 and 10, which also is aligned with the center axis of the lancet blade 14. The known position of the common centerline 100 and, hence, the path of the lancet blade 14 allows the use of a sample collection strip 102 at a predetermined position in front of the cocked lancet blade 14. The sample collection strip 102 can be prepositioned in alignment with the path of the lancet blade 14 so that the lancet blade 14 encounters the sample collection strip 102 in the same thrust in which the lancet blade 14 pierces the user's skin. In this location, the sample collection strip 102 overlies the precise location of the sampling pierce along the path of dashed line 100 so that the resulting sample of extracted blood is immediately captured on the sample collection strip 102.

In greater detail, the sample collection strip 102 is positioned at the front of the vacuum shell 94 so that the user's skin surface supports the sample collection strip 102 for the lancet blade 14 to engage the sample collection strip 102 in the same thrust as the lancet blade pierces the user's skin. The vacuum shell 94 is configured to maintain the sample collection strip in the desired position. For example, the sample collection strip 102 can be shaped as a disk, and the vacuum shell 94 might define a circular shell portion 104 of similar outlined size and shape as the disk. To better retain the sample collection strip 102 in the desired location, a mount or fastener can be used. For example as shown in FIG. 9, the vacuum shell 94 may include a mounting pin 106 that engages the sample collection strip 102 in a central mounting aperture 108. In a disk shaped sample collection strip 102, the location of the mounting aperture 108 may be at the center of the disk shape so that the sample collection strip 102 can be rotated on pin 106 while remaining within the boundaries of the shell portion 104.

Pin 106 is offset from the centerline of shell cylinder 98, although the offset preferably is smaller than or equal to the radius of the disk-shaped sampling strip 102. The center axis of the mounting pin 106 is represented by dashed line 110 in FIG. 9. The offset between dashed lines 100 and 110 is readily known. Thus, the sampling strip 102 is arranged such that the lancet 14 can pierce or otherwise engage the disk 102 between its center 108 and its outer edge or perimeter, depending upon the magnitude of the offset. An offset of one-half the radius or greater is desirable because it approaches the wider area of the sampling strip 102, near its perimeter. This positioning provides a substantial surface area of the sampling strip 102 to receive the blood sample and maintain the sample on the sampling strip.

According to the sampling method where the lancet blade passes through collection strip 102 and then pierces the user's skin, the lancet blade might sever a fleck of the collection strip 102 during advancement. It would be desirable to prevent forming such flecks and to avoid the accidental transfer of a fleck into the user's skin with the tip of the lancet blade. The chance of this sort of transfer is minimized by pre-forming a lancet passage 112 through the sampling strip 102 in the lancet blade's line of travel. A plurality of lancet blade passages 112 can be formed at equal spacing from central aperture 108, thus lying in a ring around aperture 108. The plurality of lancet blade passages 112 also may be separated by equal angles. By rotating the collection strip around aperture 108, any one of the passages 112 can be positioned in line with the lancet blade, in what can be termed the active area of the collection strip. Likewise, the passage 112 aligned with the blade can be called the active passage. Other passages 112, currently not in the active area, can be used to index the position of the collection strip so that the active passage 112 maintains alignment with the lancet blade. In FIG. 10, it is evident that dashed line 100 not only originates in alignment to the lancet blade 14, but the dashed line 100 also aligns with a pre-formed, active passage 112 in the sampling strip 102. FIG. 11 illustrates the resulting engagement between the lancet blade 14 and the pre-formed, active passage 112 in sampling strip 102.

The desired alignment of the lancet blade 14 with the active passage 112 is made possible by knowing the offset represented by the separation of dashed lines 100 and 110, as well as by locating the sampling strip in a known rotational position to place an active passage 112 in alignment with the path of the lancet blade. The rotational position of an active passage 112 is readily controlled by an indexing device acting between the vacuum shell 94 and the sampling strip 102. For example, in FIGS. 9 and 10, an indexing pin 114 is positioned to engage one of the passages 112, other than the active passage 112 that is aligned with the lancet blade.

The illustrated sampling strip 102 is configured to have a plurality of sampling areas 116 equally spaced around central mounting aperture 108. For example, in the drawings a single sampling strip is divided into eight sampling sectors 116. Each sampling area 116 is configured with a pre-formed passage 112 in the proper location to align with the lancet blade when the sampling strip 102 is suitably rotated and indexed to place a selected sampling area 116 in the active position. The single sampling strip 102 then can be used as many times as there are sectors 116 by rotating the sampling strip on the mounting pin 106, placing one sector after another into the active indexed position that is aligned with the lancet blade.

The reading wires 78 can be positioned to contact the active sampling area 116. In FIGS. 10 and 11, the reading wires 78 extend forward from the junction of ring 84 and nose 60, such that the reading wires are in a suitable position to engage the active area 116 and obtain readings of the blood sample, when present.

The foregoing is considered as illustrative only of the principles of the invention. Further, since numerous modifications and changes will readily occur to those skilled in the art, it is not desired to limit the invention to the exact construction and operation shown and described, and accordingly all suitable modifications and equivalents may be regarded as falling within the scope of the invention as defined by the claims that follow.

What is claimed is:

1. An enhanced lancet device or obtaining a blood sample, comprising:
a longitudinally elongated internal lancet housing, having a forward longitudinal end, and carrying a puncturing element at said forward longitudinal end;
wherein said puncturing element is operatively connected to said internal lancet housing and adapted for movement between a pre-actuated position in which the puncturing element is in ready condition to be driven forward, and a puncturing position in which the puncturing element is released from ready condition and driven forward on a predefined puncturing element pathway from pre-actuated position to, in use, puncture a user's skin at a puncture site to obtain a blood sample; and
wherein a biasing member is connected to the puncturing element for biasing the puncturing element to move to the puncturing position;
an external lancet housing configured to receive the internal lancet housing therein, wherein said external lancet housing is substantially pressure sealed, other than through the forward longitudinal end of the internal lancet housing;
a junction formed between the forward longitudinal end of the internal lancet housing and the forward end of said external lancet housing;
wherein said junction defines a perimeter around the forward longitudinal end of the internal lancet housing to allow forward movement of the puncturing element to puncturing position; and
means for drawing vacuum from the external lancet housing to establish suction into the internal lancet housing at the forward longitudinal end thereof for, in use, applying suction to a user's skin at said puncture site to aid in extracting a blood sample from the user.

2. The enhanced lancet device of claim 1, wherein said means for drawing vacuum comprises a tube having a first end communicating with the interior of said external lancet housing and having a second end open to the exterior of the external lancet housing, in use enabling a user to draw vacuum by mouth suction applied to said second end of said tube.

3. The enhanced lancet device of claim 1, wherein said means for drawing vacuum comprises a vacuum pump located within said external housing and communicating air to an exit port to outside the external housing.

4. The enhanced lancet device of claim 1, further comprising:
a processor located within said external housing; and
reading wires located at the forward longitudinal end of said internal lancet housing to, in use, contact an extracted blood sample at said puncture site;
wherein said reading wires are connected to said processor to enable the processor, in use, to obtain readings about a contacted blood sample.

5. The enhanced lancet device of claim 1, further comprising:
a shell mounted around said junction and extending therefrom to a forward contact edge for, in use, being pressed against a user's skin to seal said edge around said puncture site;
a sample collection strip;
means mounting said sample collection strip in said shell in a position where said predefined puncturing element pathway traverses the sample collection strip.

6. The enhanced lancet device of claim 5, wherein:
said sample collection strip is a disk having a mounting aperture at the center thereof; and
said means mounting said disk is a mounting pin carried by said shell at an offset position from said predefined puncturing element pathway, placing the predefined puncturing element pathway nearer to the perimeter of the disk than to the mounting aperture.

7. The enhanced lancet device of claim 5, wherein:
said sample collection strip is a disk having a mounting aperture at the center thereof; and
said means mounting said disk is a mounting pin carried by said shell at an offset position from said predefined puncturing element pathway;
further comprising:
indexing means holding the disk in predefined rotational position on said mounting pin with respect to said predefined puncturing element pathway;
wherein said disk predefines an opening in line with said predefined puncturing element pathway, whereby, in use, the puncturing element traverses the disk through said predefined opening.

8. The enhanced lancet device of claim 5, wherein:
said sample collection strip is a disk having a mounting aperture at the center thereof;
said means mounting said disk is a mounting pin carried by said shell at an offset position from said predefined puncturing element pathway;
further comprising:
indexing means dividing the disk into a plurality of segments at different rotational positions of the disk, and for each of said plurality of segments, when the segment is indexed in line with the predefined puncturing element pathway, holding the respective segment in a predefined position with respect to the predefined puncturing element pathway; and
wherein said disk predefines an opening in each segment, wherein when the segment is indexed in line with the predefined puncturing element pathway, the opening is in line with the predefined puncturing element pathway, whereby, in use, the puncturing element traverses each segment through the corresponding predefined opening.

9. An improved lancet device for piercing a user's skin surface to extract a blood sample, wherein the lancet device is of the type having a forward end with a spring loaded lancet holder carrying a lancet blade, a means for cocking the lancet holder to a ready position, and means for selectively releasing the cocked lancet holder from ready position to spring forward and thereby move the carried lancet blade to a forward extended position suited for piercing skin, wherein the improvement comprises:
a vacuum chamber containing the lancet device, transmitting suction through the lancet device to proximity of the lancet holder and carried lancet blade, and attached to the lancet device at said forward end by a junction encircling the position of the lancet holder;
means for drawing vacuum in said vacuum chamber;
means for selectively actuating said means for drawing vacuum to draw vacuum; and
means for guiding delivered suction in proximity to the lancet holder and lancet blade, said guiding means defining a front opening for passing the lancet blade when springing forward to extended position;
wherein said front opening is suitably configured to seal when pressed against a user's skin surface so as to deliver suction against the skin surface.

10. The improved lancet device of claim 9, wherein said means for drawing vacuum comprises a tube having a first end communicating with the interior of said vacuum chamber and having a second end open to the exterior of the vacuum chamber, in use enabling a user to draw vacuum by mouth suction applied to said second end of said tube.

11. The improved lancet device of claim 9, wherein said means for drawing vacuum comprises a vacuum pump located within said vacuum chamber and communicating air to an exit port to outside the vacuum chamber.

12. The improved lancet device of claim 9, further comprising:
a processor located within said vacuum chamber; and
reading wires located at the forward longitudinal end of said internal lancet housing to, in use, contact an extracted blood sample at said puncture site;
wherein said reading wires are connected to said processor to enable the processor, in use, to obtain readings about a contacted blood sample.

13. An improved lancet device for piercing a user's skin surface to extract a blood sample, wherein the lancet device is of the type having a forward end with a spring loaded lancet holder carrying a puncturing element, a means for cocking the lancet holder to a ready position, and means for selectively releasing the cocked lancet holder from ready position to spring forward on a puncturing element pathway and thereby move the carried puncturing element to a forward extended position suited for piercing skin, wherein the improvement comprises:
a vacuum chamber containing the lancet device, transmitting suction through the lancet device to proximity of the lancet holder and carried puncturing element, and attached to the lancet device at said forward end by a junction encircling the position of the lancet holder;
means for drawing vacuum in said vacuum chamber;
means for selectively actuating said means for drawing vacuum to draw vacuum; and
means for guiding delivered suction in proximity to the lancet holder and puncturing element, said guiding means defining a front opening for passing the puncturing element when springing forward on said puncturing element pathway to extended position.

14. The improved lancet device of claim 13, further comprising:
a shell mounted to said junction and extending forward therefrom to a forward contact edge for, in use, being pressed against a user's skin to seal said edge;
a sample collection strip;
means mounting said sample collection strip in said shell in a position where said predefined puncturing element pathway traverses the sample collection strip.

15. The improved lancet device of claim 14, wherein:
said sample collection strip is a disk having a mounting aperture at the center thereof; and
said means mounting said disk is a mounting pin carried by said shell at an offset position from said predefined puncturing element pathway, placing the predefined puncturing element pathway nearer to the perimeter of the disk than to the mounting aperture.

16. The improved lancet device of claim 14, wherein:
said sample collection strip is a disk having a mounting aperture at the center thereof; and
said means mounting said disk is a mounting pin carried by said shell at an offset position from said predefined puncturing element pathway;
further comprising:
indexing means holding the disk in predefined rotational position on said mounting pin with respect to said predefined puncturing element pathway;
wherein said disk predefines an opening in line with said predefined puncturing element pathway, whereby, in use, the puncturing element traverses the disk through said predefined opening.

17. The enhanced lancet device of claim 14, wherein:
said sample collection strip is a disk having a mounting aperture at the center thereof;
said means mounting said disk is a mounting pin carried by said shell at an offset position from said predefined puncturing element pathway;
further comprising:
indexing means dividing the disk into a plurality of segments at different rotational positions of the disk, and for each of said plurality of segments, when the segment is indexed in line with the predefined puncturing element pathway, the indexing means holds the respective segment in a predefined position with respect to the predefined puncturing element pathway; and
wherein said disk predefines an opening in each segment, wherein when the segment is indexed in line with the predefined puncturing element pathway, the opening is in line with the predefined puncturing element pathway, whereby, in use, the puncturing element traverses each segment through the corresponding predefined opening.
